(19) United States
(12) Patent Application Publication
(10) Pub. No.: US 2005/0244359 A1
(43) Pub. Date: Nov. 3, 2005

(54) Topical regulation of triglyceride metabolism

(76) Inventors: Edward Pelle, Valley Stream, NY (US); Thomas Mammone, Farmingdale, NY (US)

Correspondence Address: Karen A. Lowney, Esq.;ESTEE LAUDER COMPANIES, Suite 345 South, 155 Pinelawn Road, Melville, NY, 11747, US

(21) Appl. No.: 11/116,515

(22) Filed: Apr. 28, 2005

Related U.S. Application Data
(60) Provisional application No. 60/566,388, filed on Apr. 29, 2004.

(51) Int. Cl. A61K 7/075; A61K 7/08; A61K 7/06

(52) U.S. Cl. 424/70.22; 424/74

(57) ABSTRACT

The present invention relates to methods of topically applying compositions useful in enhancing triglyceride concentration in the lipid barrier of the skin comprising an effective amount of palmitoleic acid, or derivatives thereof, as the sole fatty acid component in combination with a pharmaceutically or cosmetically acceptable carrier. Such compositions are useful in the treatment or prevention of dry skin and conditions in which the skin's lipid barrier is defective or damaged.

### FIELD OF THE INVENTION

The present invention relates to a method of topically applying compositions to increase triglyceride production. More specifically, the invention relates to increasing triglyceride production by topical application of palmitoleic acid to improve the lipid barrier of the skin, and the acne condition.

### BACKGROUND OF THE INVENTION

Skin is typically characterized by three distinct layers, namely the stratum corneum, the epidermis and the dermis. The stratum corneum, the outermost layer, is made up of keratinized cells, surrounded by intercellular space filled with lipids. The stratum corneum provides a substantial physical barrier to penetration of most substances to the lower layers of the skin. In addition to preventing transport of substances to the other skin layers, however, this barrier also aids in prevention of water loss from the skin. Both functions are primarily attributable to the presence of the lipids in the stratum corneum.

There are two sources of the skin surface lipids making up this important barrier (i.e., the lipid barrier): sebaceous glands and the epidermis. The lipids are a diverse group of compounds, selectively comprising triglycerides, diglycerides, ceramides, free fatty acids, wax esters, cholesterol and cholesterol esters, and squalene. However, mainly, the lipids are composed of ceramide, cholesterol and free fatty acids. The quantity and composition of the skin surface lipids differ from place to place on the body, and may to some extent be related to the number of sebaceous glands in a given area of the skin. In addition, the composition of lipids varies at different depths of the epidermis. The lower epidermis has a lower quantity of sphingolipids, free sterols, cholesterol sulfate and free fatty acids. The epidermis begins to generate larger quantities of these lipids at about the level of the stratum granulosum.

About one-third of the lipid material on the surface of the skin is free and combined fatty acids. A majority of the fatty acids are straight chain monobasic acids typically no higher than C₁₈. Fatty acids on the skin are believed to be present due to the breakdown of triglycerides by microbial action, i.e., metabolic activity of lipolytic organisms. Fatty acids are important to the health of the skin as a nutrient, and unsaturated fatty acids are particularly known to possess fungicidal and bactericidal properties.

As the lipid barrier also includes triglycerides as one of its components, it is important to prevent their breakdown in order to preserve the protective nature of the lipid barrier. The lipid barrier is easily diminished by exposure to harsh detergents or soaps. The condition of the skin surface lipids may also be affected by a low level of triglycerides. It is apparent, that the quality of the skin lipid barrier can vary widely and depends on a number of different factors. Therefore, the lipid barrier may not always be adequate to perform its protective function optimally. In addition, skin that is afflicted with disease often suffers from an impaired lipid barrier. It speculated that the impaired lipid barrier is due to changes in the stratum corneum lipid composition and the organization of healthy skin.

In an attempt to compensate for a compromised lipid barrier, cosmetic compositions frequently incorporate active ingredients such as for example, hygroscopic humectants which prevent water loss, like urea or propylene glycol, or emollients, e.g., oleyl alcohol or caprylic/capric triglycerides. Certain cosmetic components may be occlusive skin conditioners, which are used to provide an “artificial” barrier; such compounds are frequently lipids which remain on the skin surface as a type of coating, and include various hydrogenated oils, waxes and butters. Although many of these products provide an effective means of stemming water loss from the skin, they have to be reapplied frequently to maintain the effect, and do not generally constitute a natural-occurring component of the stratum corneum, potentially giving rise to an unnatural or greasy feel to the skin. In addition, various pharmaceutical or cosmetic active agents are also frequently used to treat the symptoms of dry skin-associated conditions; however, in many cases, particularly with pharmaceutical agents, the treatments themselves may cause undesirable side effects on the area of skin being treated, while ultimately results in no actual repair of the lipid barrier.

Little is thoroughly understood and the literature is sparse on free fatty acids in the stratum corneum even though biological activity has long been attributed to fatty acids and related compounds. The importance of long-chain fatty acids was reported in stratum corneum and particularly in the plantar stratum corneum where C₁₈ and C₂₄ predominate. Quantitative analytical data regarding fatty acids in human stratum corneum has been provided to further the understanding of free fatty acids in normal plantar stratum corneum. See Nicollier, M., et al., “Free Fatty Acids and Fatty Acids of Triacylglycerols in Normal and Hyperkeratotic Human Stratum Corneum,” J. Invest. Derm., vol. 87, pp. 68 to 71 (1986). In another study, a diet of a myristoleic acid/palmitoleic acid mixture containing myristic acid has been suggested as being particularly capable of increasing the level of cholesterol in swine subjects. See Smith et al., “A Diet Containing Myristoleic Plus Palmitoleic Acids Elevates Plasma Cholesterol in Young Growing Swine,” Lipids, vol. 31, pp. 849-858 (1996). The addition of myristic acid to the fatty acid mixture resulted in a higher plasma myristic acid level, and plasma myristoleic and palmitoleic acid levels increased in diets enriched with the fatty acid mixture. A similar effect was found when diets were enriched with palmitic acid and linoleic acid. Stearic acid, however, was not found to be affected by diet. Diets enriched with stearic acid were believed, however, to result in elevated levels of plasma linoleic acid. In this study, it was noted that while cholesterol concentrations rose, plasma triglycerides were unaffected by dietary fatty acids. In yet another study, the application of safflower oil (linoleic acid) to animal skin was found to increase the linoleic acid content of plasma triglyceride. Bohles et al., “Reversal of Experimental Essential Fatty Acid Deficiency by Cutaneous Administration of Safflower Oil,” American Journal of Clinical Nutrition, vol. 29, pp. 398-401 (1976).

Further, lipid compositions for cosmetic products are disclosed in U.S. Pat. No. 5,653,966 containing oils that contain a mixture of fatty acids with palmitoleic acid. However, it has not heretofore been recognized, that palmitoleic acid when applied, as the sole fatty acid component, directly to the skin, would have an effect in increasing triglyceride concentration in the lipid barrier of the skin. Fatty acids that occur naturally in fats and oils as glyceryl esters of fatty acids contain mixtures of fatty acids. Fatty acids can be esterified with glycerin to yield mono-, di-, and triglycerides. However, it has now been surprisingly found that palmitoleic acid, when applied topically to the skin in an effective amount, is capable of increasing the concentration of triglycerides, one of the naturally occurring stratum corneum lipid components, as well as consequently enhancing the protective nature of the stratum corneum per se.

The present invention provides a useful substitute for the daily application of skin conditioning agents, or harsh topical active agents. There is thus provided a gentle type of cosmetic or pharmaceutical composition which functions by enhancing the skin's own lipid barrier function, and resulting in a more natural means of preventing dry skin and other undesirable results of a deficient lipid barrier such as the acne condition.

### SUMMARY OF THE INVENTION

The present invention relates to a method of applying topical compositions comprising as a sole fatty acid component, palmitoleic acid in an amount effective to increase triglyceride levels in the skin. The palmitoleic acid is in combination with a cosmetically or pharmaceutically acceptable carrier. The invention also relates to a method for stimulating triglyceride synthesis in the lipid barrier of the skin which comprises applying to the skin an effective amount of palmitoleic acid. As the lipid barrier is a key factor in maintaining the quality and moisture of skin, the topical application of palmitoleic acid is useful in improving overall skin condition, and in the prevention or treatment of a variety of dry skin conditions generally, and specific skin conditions, such as for example the acne condition, in which the natural lipid barrier is compromised or absent.

The topical compositions comprise a fatty acid component of solely palmitoleic acid in a cosmetically or pharmaceutically acceptable carrier. The palmitoleic acid is the only fatty acid in the composition and is not part of a mixture as in, for example, oils, fats and waxes.

### DETAILED DESCRIPTION OF THE INVENTION

The topical application of a single small molecular weight fatty acid to the skin is capable of stimulating triglyceride production in a way that mimics the natural production of triglycerides at the surface of the skin. The particular fatty acid is palmitoleic acid and it is also known as hexadecenoic acid (16:1). Triglycerides are produced naturally by the skin to provide moisturization and barrier protection. It has been reported in “Free Fatty Acids and Fatty Acids of Triacylglycerols in Normal and Hyperkeratotic Human Stratum Corneum”, Nicollier, Monique, Ph.D., et al., Soc. Inv. Derm., vol. 87, no. 1, pp. 68 to 71 (1986), that normal human stratum corneum was found to contain about 0.7 percent palmitoleic acid and cis-9-palmitoleic acid (C_{16:1}). It is also reported that palmitic acid and oleic acid are found to be present in the stratum corneum at about 9.2 and 11.9 percent. Thus, these acids are more prevalent in the natural stratum corneum than palmitoleic acid. The discovery of the present invention is particularly surprising because the increase in triglycerides found with palmitoleic acid is greater than the increase found with oleic acid. Comparatively, oleic acid, when used alone in the same treatment regimen, has little effect.

In view of this activity, the present invention has found that palmitoleic acid is a very useful component for cosmetic and/or topically delivered pharmaceutical compositions by increasing the triglyceride concentration of the lipid barrier. It will be understood that throughout the specification and claims, where the term “palmitoleic acid” is used this term also encompasses the free fatty acid forms of palmitoleic acid. Forms of palmitoleic acid include, but are not limited to, 2-hexadecenoic acid, 3-hexadecenoic acid, 6-hexadecenoic acid, 9-hexadecenoic acid, 10-hexadecenoic acid, and 11-hexadecenoic acid. Palmitoleic acid is an unsaturated long chain free fatty acid which is surprisingly readily available for the formation of triglycerides in the lipid barrier of the skin. It is a naturally occurring component of complex mixtures of fatty acids in human, animal and vegetable fats and waxes. Particularly, 6-hexadecenoic acid, and 10-hexadecenoic acid are noted as being present in adipose tissue. In the present invention, the fatty acid component is solely palmitoleic acid that is substantially pure, i.e., at least 70% pure, preferably at least 80% pure and more preferably at least 90% pure. They are commercially available from for example Sigma-Aldrich, Saint Louis, Mo.

Corn oil provides a source of mixed triglycerides, like other vegetable oils such as, for example, palm kernel oil, coconut oil, soybean oil, sunflower oil, safflower oil, cocoa butter, and the like. Therefore, in order to achieve the increase in triglycerides in the skin, the palmitoleic acid is separated from these types of oils. A combination of triglycerides, as is found in naturally occurring oils, waxes, and fats, has not heretofore been known to have the effect on stimulating triglyceride production on the skin that palmitoleic acid alone has. It is surprising to find that the present invention containing solely palmitoleic acid has such an effect. As used in the present specification and claims, “triglyceride enhancing effective amount” is intended to indicate an amount capable of increasing the production or metabolism of triglycerides by at least 5 percent, preferably 10 percent and more preferably 20 percent when compared with untreated skin in the same location. Results are achievable at even greater percentages up to between about 200 to 500 percent. Alternatively, the efficacy of palmitoleic acid is evaluated by its ability to strengthen the lipid barrier as indicated by its effect on an epidermal equivalent. In the present context, an amount of palmitoleic acid is considered effective if it enhances lipid barrier strength after at least 5 days of treatment, preferably after 3 days, and more preferably after 1 day. In formulating the compositions of the present invention containing palmitoleic acid as the sole fatty acid, palmitoleic acid is incorporated in a triglyceride enhancing effective amount of from about 0.001 to 50.0 percent by weight of the composition, preferably about 0.005 to 10.0 percent by weight, more preferably about 0.01 to about 1.0 percent.

It is suggested, as an example, that topical application range from about every other day to about 1 to 3 times daily, preferably from about once daily to about 2 times daily, most preferably about once or twice per day. Application of the compositions of the present invention, however, can be chronic. By “chronic” application, it is meant herein that the period of topical application may be over the lifetime of the user, preferably for a period of at least about one month, more preferably from about three months to about twenty years, more preferably from about six months to about ten years, more preferably still from about one year to about five years, thereby resulting in the achievement of increasing the concentration of triglycerides in the lipid barrier. For example, if the compositions of the present invention are in the form of a lip product, application can be on a regular daily basis. The effect in increasing triglycerides can last for up to about one day.

The method and frequency of application of the compositions will vary depending upon the form of the composition. With respect to the method for application, the composition will generally be applied to the skin in the same manner as one would apply other compositions of the same type and form, e.g., as a cream or lotion to be applied for increasing triglycerides in the skin. As to the frequency, for treatment of existing dry skin conditions or other conditions associated with a defective or damaged barrier, or a fatty acid deficiency, the composition can be applied on an as-needed basis, for example, until the condition is improved. When used in combination with other active agents, as outlined below, the application frequency will be determined according to the usual pattern for topical application of the other active. It will be recognized by those skilled in the art that the treatment regimen employed can vary depending upon the individual's biological profile pertaining to triglyceride levels in the lipid barrier.

For topical application, palmitoleic acid can be formulated with a variety of cosmetically and/or pharmaceutically acceptable carriers. The term “pharmaceutically and/or cosmetically acceptable carrier” refers to a vehicle, for either pharmaceutical or cosmetic use, which vehicle delivers the active components to the intended target and which will not cause harm to humans. As used herein, “pharmaceutical” or “cosmetic” will be understood to encompass both human and animal pharmaceuticals or cosmetics. Useful carriers include, for example, ethanol, ethylene glycol, propylene glycol, butane-1,3-diol, or media cell culture. Methodology and components for formulation of cosmetic and pharmaceutical compositions are well known, and can be found, for example, in Remington's Pharmaceutical Sciences, Eighteenth Edition, A. R. Gennaro, Ed., Mack Publishing Co., Easton, Pa., 1990. The carrier may be in any form appropriate to the mode of delivery, for example, solutions, colloidal dispersions, emulsions (oil-in-water or water-in-oil), suspensions, creams, lotions, gels, foams, mousses, sprays and the like.

The formulation, in addition to the carrier and the free fatty acid component, also can comprise other components that may be chosen depending on the carrier and/or the intended use of the formulation. Additional components include, but are not limited to, water soluble colorants (such as FD&C blue #1); oil soluble colorants (such as D&C green #6); water soluble sunscreens (such as Eusolex 232); oil soluble sunscreens (such as octyl methoxycinnamate); particulate sunscreens (such as zinc oxide); antioxidants (such as BHT); chelating agents (such as disodium EDTA); emulsion stabilizers (such as carbomer); preservatives (such as methylparaben); fragrances (such as pinene); flavoring agents (such as sorbitol); humectants (such as glycerine); waterproofing agents (such as PVP/eicosene copolymer); water soluble film-formers (such as hydroxypropyl methylcellulose); oil-soluble film formers (such as hydrogenated C-9 resin); cationic polymers (such as Polyquaternium 10); anionic polymers (such as xanthan gum); vitamins (such as tocopherol); and the like.

The therapeutic/cosmetic uses of the present compositions are numerous, namely treatment or prevention of any condition in which the natural lipid barrier of the skin or the lip is at risk, deficient or damaged. For example, the palmitoleic acid compositions can be used in prevention or treatment of dry skin conditions generally, or specific dry skin conditions, such as result from regular exposure to detergents, soaps and hot water; seasonal exposure to harsh weather conditions, e.g., cold, wind and/or sun; occupational exposure to harsh chemicals or other drying or damaging agents; or pathological conditions such as eczematous dermatides, psoriasis, ichthyoses, xerosis and the like. It is also well-known that dry skin is commonly associated with aging (both intrinsic and photoaging), and the palmitoleic acid compositions can be used in prevention of further damage to aging skin, or treatment and/or reversal of already present damage. The compositions can also be used in the treatment of a defective skin barrier, such as that which occurs on the soles of the feet, and the palms of the hands, where the stratum corneum is very thick, but the lipid barrier is poor. In addition, defective skin barriers frequently occur in association with burns, wounds, blisters, stasis ulcers and bedsores; such injuries can be expected to benefit from application of the compositions of the invention.

Given the various uses of the triglyceride-enhancing formulations, therefore, it will be understood that the formulations also can comprise other components that are chosen depending on the carrier and/or the intended use of the formulation. Examples of additional components include, but are not limited to, water soluble colorants (such as FD&C Blue #1); oil soluble colorants (such as D&C Green #6); water soluble sunscreens (such as Eusolex 232); oil soluble sunscreens (such as Octyl Methoxycinnamate); particulate sunscreens (such as Zinc Oxide); antioxidants (such as BHT); chelating agents (such as Disodium EDTA); emulsion stabilizers (such as carbomer); preservatives (such as Methyl Paraben); fragrances (such as pinene); emollients (such as petrolatum); flavoring agents (such as sorbitol); humectants (such as glycerine); waterproofing agents (such as PVP/Eicosene Copolymer); water soluble film-formers (such as hydroxypropyl methylcellulose); oil-soluble film formers (such as Hydrogenated C-9 Resin); cationic polymers (such as Polyquatemium 10); anionic polymers (such as xanthan gum); and the like.

The present compositions may also be beneficially combined with other active agents which are used for skin treatment (both cosmetic and pharmaceutical), or which are routinely applied topically. Examples of such active agents which may be usefully combined with palmitoleic acid include, but are not limited to, those that improve or eradicate age spots, keratoses and wrinkles, analgesics, anesthetics, anti-acne agents, antibacterials, antiyeast agents, antifungal agents, antiviral agents, antidandruff agents, antidermatitis agents, antipruritic agents, antiemetics, antimotion sickness agents, anti-inflammatory agents, antihyperkeratolytic agents, anti-dry skin agents, antiperspirants, antipsoriatic agents, antiseborrheic agents, hair conditioners and hair treatment agents, antiaging agents, antiwrinkle agents, antiasthmatic agents and bronchodilators, antihistamine agents, skin lightening agents, depigmenting agents, wound-healing agents, vitamins, corticosteroids, tanning agents, or hormones. More specific examples of useful active agents include retinoids, topical cardiovascular agents, clotrimazole, ketoconazole, miconozole, griseofulvin, hydroxyzine, diphenhydramine, pramoxine, lidocaine, procaine, mepivacaine, monobenzone, erythromycin, tetracycline, clindamycin, meclocyline, hydroquinone, minocycline, naproxen, ibuprofen, theophylline, cromolyn, albuterol, retinoic acid, 13-cis retinoic acid, hydrocortisone, hydrocortisone 21-acetate, hydrocortisone 17-valerate, hydrocortisone 17-butyrate, betamethasone valerate, betamethasone diproprionate, DHEA and derivatives thereof, triamcinolone acetonide, fluocinonide, clobetasol, proprionate, benzoyl peroxide, crotamiton, propranolol, promethazine, vitamin A palmitate, vitamin E acetate and mixtures thereof. The amount of active agent to be used in any given formulation is readily determined in accordance with its usual dosage.

The formulations of the present invention can be used for both cosmetic and pharmaceutical applications. For example, the formulation may take the form of a cosmetic, such as a lipstick or lip gloss, wherein the triglyceride-enhancing compositions potentially have both a therapeutic and maintenance effect. In addition, the formulation may be completely therapeutic, for example, intended to be used in application to lips which are in need of lipid barrier repair or fortification due to the damaging effects of diseases, chronic or acute conditions, or environmental insult. Examples of conditions which can benefit from application of such combinations include, but are not limited to, ulcers, blisters, herpes virus infections, severe chapping, or burns. Methods and/or regimens for application of the lip products of the invention are in accordance with the normal usage of products of similar type, i.e., lipsticks, lip balms, ointments, etc. Additional uses of the formulations of the invention will be readily apparent to those skilled in the art.

For compositions of the present invention that are used and applied to the lip area, the base may be of any type which is appropriate for use on the lips and around mucous membranes. The vehicle may take the form of creams, sticks, gels and the like. A particularly preferred form of base, however, will be a waxy base, more typically an anhydrous waxy base, as is usual for most products intended for application to the lips. Methods for formulating such wax based products are well known in the art. Briefly, a typical waxy base contains one or more waxes, one or more oils, and one or more surfactants to aid in dispersing the components. “Waxes” as used herein intended to comprise not only waxes in the traditional sense, i.e., those plant, animal or mineral waxes containing primarily esters of higher fatty acids and alcohols, free higher acids and alcohols, and saturated hydrocarbons, but also synthetic resinous products having a wax-like, i.e., hard, brittle, relatively non-greasy, texture, such as silicone waxes. Examples of suitable waxes for use in the wax base include, but are not limited to, carnauba wax, candelilla wax, beeswax, synthetic wax, shellac wax, spermaceti, lanolin wax, ozokerite, bran wax, ceresin wax, bayberry wax, paraffin, rice wax and jojoba wax. “Oils” as used herein encompass not only naturally occurring plant, animal and mineral oils, but also oil-like emollients, such as fatty esters, fatty alcohols, and silicone oils. The surfactant may be any that is routinely used in this type of product. Examples of components useful in formulating cosmetic bases are found, for example, in the International Cosmetic Ingredient Handbook, CTFA, 1996, contents of which are incorporated herein by reference.

The invention will be further understood by reference to the following non-limiting examples.

### EXAMPLE

Comparison of Palmitoleic Acid Activity with Oleic Acid Activity.

A set of living skin samples, epidermal equivalents, obtained from MatTek, is tested with a sample of about 0.1 percent palmitoleic acid and about 0.1 percent oleic acid. A media is prepared of a transwell membrane at the air/liquid interface and is cultured for three days. At the end of the incubation period, each skin square is homogenized and its triglycerides extracted in chloroform/methanol (2:1). This is followed by HPTLC analysis.

The HPTLC results show an increase in triglycerides as a function of time and dose. After one day of application it is surprising to find the increase in triglyceride concentration in the epidermal equivalents treated with palmitoleic acid. The comparative testing confirms a time and dose-dependent increase in triglyceride production in the living skin models treated with palmitoleic acid. The net intensity of oleic acid is 859. In contrast, palmitoleic acid has a net intensity of 1324 and 1212, an average of 1268 net intensity. This indicates about a 400% increase in triglyceride production using the compositions of the present invention, and demonstrates that the increase in triglycerides is due to a stronger activity of palmitoleic acid in comparison with oleic acid. According to this data, palmitoleic acid increases triglyceride production more rigorously than oleic acid. These results indicate that the treatment including palmitoleic acid as the sole fatty acid surprisingly increases the triglyceride concentration, and have an effect on the topical regulation of triglyceride metabolism.

1. A topical cosmetic or pharmaceutical composition for enhancing the level of triglycerides on the epidermal surface comprising a fatty acid component comprising solely palmitoleic acid in a cosmetically or pharmaceutically acceptable carrier.

2. The composition of claim 1 wherein the palmitoleic acid is extracted from an oil selected from the group consisting of palm oil, rice bran oil, sesame oil, avocado oil, macadamia oil, apricot kernel oil, and olive oil.

3. A method for increasing triglyceride concentration on skin comprising the step of topically applying a composition comprising a triglyceride enhancing effective amount of palmitoleic acid in a cosmetically or pharmaceutically acceptable carrier.

4. The method of claim 1 in which the effective amount of palmitoleic acid is from about 0.001 to about 50 percent by weight of the composition.

5. The method of claim 1 in which the effective amount of palmitoleic acid is from about 0.005 to about 10.0 percent by weight of the composition.

6. The method of claim 1 in which the effective amount of palmitoleic acid is from about 0.01 to about 1.0 percent by weight of the composition.

7. The method of claim 1 wherein the composition further comprises an additional cosmetic and/or pharmaceutical topically active agent.

8. A method for improving skin or lip condition which comprises applying to the skin the composition of claim 1 by increasing triglyceride synthesis in the skin.

9. A method for treatment or prevention of dry skin or acne condition which comprises applying to the skin the composition of claim 1 to increase lipid synthesis.

11. A method for strengthening the lipid barrier in skin which comprises applying the composition of claim 1.

12. A cosmetic or pharmaceutical composition comprising a fatty acid component that is solely palmitoleic acid and present in a triglyceride enhancing effective amount in an acceptable carrier.
